(19) United States
(12) Patent Application Publication
(10) Pub. No.: US 2004/0147616 A1
(43) Pub. Date: Jul. 29, 2004

(54) Curative medicine and tools for the athlete's foot with medicine liquid and high concentration alcohol

(76) Inventor: Takashi Furukawa, Nagoya (JP)

Correspondence Address: Takashi Furukawa, 186-1 Kamiyama Yatomi-Cho, Mizuho-Ku, Nagoya, 467-0037, JP

(21) Appl. No.: 10/759,292

(22) Filed: Jan. 16, 2004

(30) Foreign Application Priority Data
Jan. 22, 2003 (JP) 2003-50080

(51) Int. Cl. A61K 31/045

(52) U.S. Cl. 514/724

(57) ABSTRACT

The athlete's foot had a concept of that which cannot be healed up very easily. It is because that the medical treatment method is not suitable. I have invented the immersing medical treatment method by the time unit with the retail medicine from the manufacture companies and rubbing alcohol to permeate into dermis.

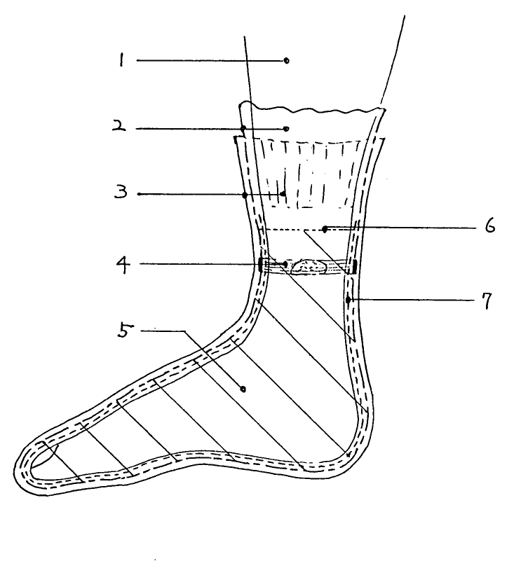

### BACKGROUND OF THE INVENTION

This invention uses the athlete's foot medicine and high concentration alcohol of the conventional marketing, and relates to the tools and the medical treatment method and using the prepared thing and its simple substance There is nothing that can carry out perfect recovering of the athlete's foot, although the medicine manufacture companies in the world have made much athlete's foot medicine from the former. The present condition is that the man in the world moreover also desires extermination and perfect recovering for athlete's foot for a short period of time now.

### SUMMARY OF THE INVENTION

It relates to finding the way that the man in the world was in trouble for years and full medical treatment was studied. It acts with a little athlete's foot medicine in high concentration alcohol, the leg was immersed per time, and it invented perfect recovering for the athlete's foot bacillus for a short period of time.

### BREEF DESCRIPTION OF THE DRAWINGS

This is explanation for wearing bags and socks on feet witch are familiarized with legs, and puts either of the A-B-C in into a bag.

1) Foot

2) Waterproof bag

3) Socks which keep the liquid

4) Binding up the bag

5) Putting the liquid to the bag

6) The line which indicates the liquid comes up

### DETAILED DESCRIPTION OF THE INVENTION

This invention is explained in detail. An athlete's foot has the concept what is not cured and everybody has an idea near abandonment. even when it seemed that medicine is applied and it healed up, I thought that it came out again This invention tried to act as perfect recovering of the athlete's foot for a short period of time. The medical treatment method is described below.

A) The retail medicine from the manufacture companies—10˜20 Vol. % to rubbing alcohol—into the rubbing alcohol of—79 Vol. %—make the athlete's foot curative medicine liquid diluted 5 to 10 times from the high concentration alcohol which makes order the optimal.

B) Prepare the bag with which the liquid will not carried out during the legs being immersed

C) Put legs into D, and wear, binding bags and extracting socks or the bags of elasticity on it.

F) Thin socks may be worn into a bag.

G) Each of the A-B-C are put into the bag, and put over than ankles or over than the medical treatment range, and the skin of legs will become like fish scales, and continue about one week twice in the morning and in evening for one day, and it separates being immersed for several minutes to 1 hour or more, it will be stopped. It finishes when they separate. And at the time of a serious illness, and it will need to repeat the same way again.

H) Disinfect inside of the shoes every day with the rubbing alcohol.

I) Disinfected socks must be used each time. It is for preventing re-infection on foot.

J) Also disinfected and sterilize the mat in the bathroom and others in the same way are needed. The above medical treatment methods are for extinguishing athlete's foot bacillus from the feet and life circumference so that a curative medicine's may be made to permeate to the dermis to which an athlete's foot bacillus breeds, an athlete's foot bacillus's may be killed and it may not recur.

What claimed are:

A) The retail medicine from the manufacture companies which melts into the rubbing alcohol of—79 VOI %—make the athlete's foot curative medicine liquid diluted 5 to 10 times from the high concentration alcohol which makes order the optimal.

B) Preparing the required quantity of the following medical treatment method for the athlete's foot medicine of each medicine manufacture companies.

C) Preparing the high concentration rubbing alcohol 79 VOI %—which makes order the optimal

D) Preparing the bags liquid leak of the leg is not carried out for being immersed.

E) Binding up the bags in which the legs immersed. Preparing socks or the bags of elasticity.

F) Putting each of the A-B-C into the bag, and steeping over than ankles or over than the medical treatment range till the skin of legs will become like fish scales, and continue about one week twice in the morning and in evening for one day till it separates being immersed for several minutes to 1 hour or more, then being stopped. And finish when they separate. And at the time of a serious illness, it will be needed repeating the same thing again.
